(19) United States
(12) Patent Application Publication
(10) Pub. No.: US 2024/0197169 A1
(43) Pub. Date: Jun. 20, 2024

(54) METHOD FOR DETERMINING ACCURATE VALUES OF REFRACTIVE FEATURES OF AN EYE OF A SUBJECT IN NEAR ANDOR INTERMEDIARY VISION CONDITIONS AND ASSOCIATED METHOD

(71) Applicant: Essilor International, Charenton-Le-Pont (FR)

(72) Inventor: Martha HERNANDEZ-CASTANEDA, Paris (FR)

(73) Assignee: Essilor International, Charenton-Le-Pont (FR)

(21) Appl. No.: 18/555,621

(22) PCT Filed: Apr. 22, 2022
(86) PCT No.: PCT/EP2022/060759

§ 371 Date: Oct. 16, 2023

(30) Foreign Application Priority Data
Apr. 28, 2021 (EP) 21305548.6

(51) Int. Cl. A61B 3/028; A61B 3/00; A61B 3/032; A61B 3/08; A61B 3/113

(52) U.S. Cl. CPC A61B 3/0285; A61B 3/0083; A61B 3/032; A61B 3/08; A61B 3/113

(57) ABSTRACT

A method using an optometry device having a refraction test unit with a first optical refraction element adapted to provide different vision correction powers to a first eye of the subject along a first optical axis and a second optical refraction element adapted to provide different vision correction powers to the second eye of the subject along a second optical axis. The method includes adjusting the relative position of the subject and the refraction test unit, determining a vertex distance between the first or second optical refraction element and the first or second eye of the subject, determining a value of a parameter representative of an accommodation feature of the eyes of the subject, determining preliminary values of the refractive features of the eyes of the subject in near and/or intermediary vision conditions, checking binocular visual perception of the subject, and determining a final visual acuity of the subject.

### TECHNICAL FIELD OF THE INVENTION

The invention relates to a method for determining accurate values of refractive features of an eye of a subject in near and/or intermediary vision conditions.

This method may especially be used in a method for determining a complete set of values of refractive features of an eye of a subject

### BACKGROUND INFORMATION AND PRIOR ART

Known devices and methods for binocular testing of the eyes of a subject are usually dedicated to testing the eyes in far vision conditions. In far vision conditions, the image shown to the subject to test his binocular far vision is usually placed at over 1 meter from the subject's eyes.

However, the refraction features of the eyes in near and/or intermediary vision conditions may be different from the refraction features in far vision. This may be due, among other things, to the fact that the accommodation and convergence of the eyes are different in near and/or intermediary vision conditions.

### SUMMARY OF THE INVENTION

Therefore one object of the invention is to provide a new method allowing determining precisely the refraction features of the eyes of the subject in near and/or intermediary vision conditions. In particular, the new method proposed allows determining the refraction features of the eyes of the subject in near and/or intermediary vision conditions while keeping both of the eyes of the subject open.

The above object is achieved according to the invention by providing a method for determining accurate values of refractive features of an eye of a subject in near and/or intermediary vision conditions, using an optometry device having a refraction test unit with a first optical refraction element adapted to provide different vision correction powers to a first eye of the subject along a first optical axis and a second optical refraction element adapted to provide different vision correction powers to the second eye of the subject along a second optical axis,
comprising the following steps:
a) adjusting the relative position of the subject and said refraction test unit so that the pupils of his eye are aligned with said first and second optical axes in near and/or intermediary vision conditions,
b) determining a vertex distance between said first or second optical refraction element and said first or second eye of the subject in near and/or intermediary vision conditions,
c) determining a value of a parameter representative of an accommodation feature of the eyes of the subject in near and/or intermediary vision conditions,
d) determining preliminary values of said refractive features of the eyes of the subject in near and/or intermediary vision conditions, comprising at least a preliminary value of the spherical refraction of each eye in near and/or intermediary vision conditions and a preliminary value of the cylindrical refraction of each eye in near and/or intermediary vision conditions,
e) checking binocular visual perception of the subject by adding to the vision correction powers provided to the eyes an added predetermined spherical power,
f) determining a final visual acuity of the subject in near and/or intermediary vision conditions, and
g) determining said accurate values of refractive features of an eye of a
subject in near and/or intermediary vision based on the results of the previous steps, wherein, in said step d), the following steps are performed in this order:
   measuring a first value of spherical refraction of each of the eyes of the subject,
   measuring said preliminary value of cylindrical refraction of each of the eyes,
   measuring a second value of spherical refraction of each of the eyes of the subject, said preliminary value of spherical refraction of each of the eyes of the subject being determined based on said second value.

This specific sequence of steps makes it possible to determine both spherical and cylindrical refraction features of both eyes of the subject in an accurate yet fast manner, in near and/or intermediary vision conditions.

The method of the invention is preferably applied to each eye in order to determine the features of both eyes.

The refractive features comprise at least one of the following: spherical refraction, cylindrical refraction and axis.

In the following, near vision conditions will correspond to a distance between the eyes of the subject and a visual target of less than or equal to 50 centimeters, preferably comprised between 25 and 50 centimeters. The intermediary vision conditions will correspond to a distance between the eyes of the subject and a visual target over 50 centimeters, less than or equal to 100 centimeters, preferably comprised between 50 and 100 centimeters. In near and intermediary vision conditions, the gaze direction of the eyes of the subject may be straight ahead, approximately horizontal, or inclined downwards, preferably by an angle comprised between 10 and 50 degrees relative to a horizontal direction.

These distances are achieved thanks to a display system of said optometry device allowing displaying a visual target at a predetermined distance from said first and second optical refraction elements of said refraction test unit.

According to other advantageous and non-limiting features of the method of the invention:
in step a), a camera oriented towards the eyes of the subject, an eye tracking device or a support for the head of the subject is used;
in step b), at least a profile image of the subject placed in front of the refraction test unit is acquired, and the distance between each eye and the corresponding optical refraction test element is deduced from this profile image;
before step d), a preliminary visual acuity test of the eyes of the subject is performed with initial spherical power and/or cylindrical power and axis of each optical refraction element determined based on refraction features of a current optical equipment of the subject or on the refraction features of the eyes of the subject in far vision conditions;
in step c), a binocular accommodation test is performed by presenting each of the eyes of the subject with an identical target placed at near or intermediary vision optical distances from the eyes of the subject and modifying the spherical powers of each of the first and second optical refraction elements to determine the minimum spherical powers of the first and second optical refraction element for which the subject accommodates on the target;
each step of measuring said first value of spherical refraction of each of the eyes of the subject, measuring said preliminary value of cylindrical refraction of each of the eyes, and measuring said second value of spherical refraction is performed by testing both eyes of the subject simultaneously in binocular vision, or by testing each eye of the subject separately from the other while maintaining a binocular vision;
said first and second values of spherical refraction of each of the eyes of the subject are determined by testing both eyes simultaneously in binocular vision; or
said first value of spherical refraction of each of the eyes of the subject is determined by testing both eyes simultaneously in binocular vision and said second value of spherical refraction of each of the eyes of the subject is determined by testing each eye separately from the other while maintaining a binocular vision; or
said first value of spherical refraction of each of the eyes of the subject is determined by testing each eye separately from the other while maintaining a binocular vision and said second value of spherical refraction of each of the eyes of the subject is determined by testing both eyes simultaneously in binocular vision; or
said first and second values of spherical refraction of each of the eyes of the subject are determined by testing each eye separately from the other while maintaining a binocular vision;
said first and second values of spherical refraction and said preliminary value of cylindrical refraction of each of the eyes of the subject are determined by testing each eye separately from the other while maintaining a binocular vision and said first values of spherical refraction, said preliminary value of cylindrical power and axis and said second value of spherical refraction are obtained by alternating measures on the first and second eyes of the subject;
in said step d) for determining said preliminary value of cylindrical refraction of each of the eyes, each of the eyes is tested separately from the other while maintaining a binocular vision;
said preliminary values of the spherical and cylindrical refraction of the eye comprise a spherical power Se and a cylindrical power Ce having an orientation represented by an angle Ae or an equivalent sphere Me equal to the spherical power Se plus half of the cylindrical power Ce and refractive powers J0e, J45e of two Jackson crossed cylinders lenses representative of the cylindrical refractive power features of the eye;
between steps d) and e), a step of adjusting the binocular balance of the eyes of the subject to obtain adjusted values of said spherical and cylindrical refraction of the eyes in near or intermediary vision conditions is performed, and in step e) said binocular visual perception of the subject is checked by providing the eyes with vision correction powers of each of the first and second optical refraction elements equal to said adjusted values of spherical and cylindrical refraction with an added predetermined spherical power;
in step d), first and second test images are provided to said first and second eyes of the subject, comprising images of realistic activities carried out in near vision having a plurality of peripheral image components displayed stereoscopically to be perceived in three dimensions by the subject;
in each step where each of the eyes is tested separately from the other while maintaining a binocular vision:
   a tested-eye test image with target is provided to the tested eye, comprising images of realistic activities carried out in near vision having a plurality of peripheral image components and a test target displayed in the center of the test image, and
   a non-tested-eye test image is provided to the other eye comprising similar images of realistic activities carried out in near vision having a plurality of peripheral image components and no test target displayed in the center or the same test target than displayed on the tested-eye test image, said test target of the non-tested-eye test image being displayed with a contrast inferior to the contrast of the test target of the tested-eye test image, the peripheral image components of the two test images being stereoscopically displayed;
   said non-tested-eye test image or at least the test target of the non-tested-eye test image is displayed with a contrast inferior to half of the contrast of the tested-eye test image or to half the contrast of the test target of the tested-eye test image;
   in each step where both eyes are tested simultaneously, a test image with target is provided to each eye, comprising images of realistic activities carried out in near or intermediary vision having a plurality of peripheral image components displayed stereoscopically, each component of said plurality of peripheral image components being displayed with a specific disparity between the images provided to both eyes of the subject that is different from the disparity associated to other components of said plurality of peripheral image components;
   in each step where both eyes are tested simultaneously in binocular vision, a test image is provided to each eye, comprising images of realistic activities carried out in near vision having a plurality of peripheral image components displayed stereoscopically and a test target displayed in the center of the test image;
   each component of said plurality of peripheral image components is displayed with a specific disparity between the images provided to both eyes of the subject that is different from the disparity associated to other components of said plurality of peripheral image components;
   before performing steps a) to g), the refractive features of the eye in far vision conditions are determined, and a test for determining the need for correcting astigmatism of the eye is performed, comprising measuring the visual acuity of the eye with test images having different contrasts;
   during said steps a) to f), said first and second optical axes of said optical refraction elements are inclined downwards.

The invention also relates to a method for determining a complete set of values of refractive features of an eye of a subject in far and near and/or intermediary vision conditions, comprising a determination of accurate values of refractive features of an eye of a subject in far vision conditions and a determination of accurate values of refractive features of an eye of a subject in near and/or intermediary vision conditions as described above.

Advantageously, said determination of accurate values of refractive features of an eye of a subject in far vision conditions comprises the following steps:
A) adjusting the relative position of the subject and said refraction test unit so that the pupils of his eye are aligned with said first and second optical axes in far vision conditions,
B) determining a vertex distance between said first or second optical refraction element and said first or second eye of the subject in far vision conditions,
C) determining a value of a parameter representative of an accommodation feature of the eyes of the subject in far vision conditions or altering said accommodation of the eyes of the subject,
D) determining preliminary values of said refractive features of the eyes of the subject in far vision conditions, comprising at least a preliminary value of the spherical refraction of each eye and a preliminary value of the cylindrical refraction of each eye,
E) checking binocular visual perception of the subject by adding to the vision correction powers provided to the eyes an added predetermined spherical power,
F) determining a final visual acuity of the subject in near and/or intermediary vision conditions, and
G) determining said accurate values of refractive features of an eye of a subject in far vision based on the results of the previous steps,
wherein in said step d), the following steps are performed in this order:
   measuring a first preliminary value of spherical refraction of each of the eyes of the subject,
   measuring said preliminary value of cylindrical refraction of each of the eyes,
   measuring a second preliminary value of spherical refraction of each of the eyes of the subject, said preliminary value of spherical refraction of each of the eyes of the subject being determined based on said second preliminary values;
   the refractive features of said eye in far and near vision conditions are determined within 24 hours.

### DETAILED DESCRIPTION OF EXAMPLE(S)

The following description with reference to the accompanying drawings will make it clear what the invention consists of and how it can be achieved. The invention is not limited to the embodiment/s illustrated in the drawings. Accordingly, it should be understood that where features mentioned in the claims are followed by reference signs, such signs are included solely for the purpose of enhancing the intelligibility of the claims and are in no way limiting on the scope of the claims.

On the appended drawings:

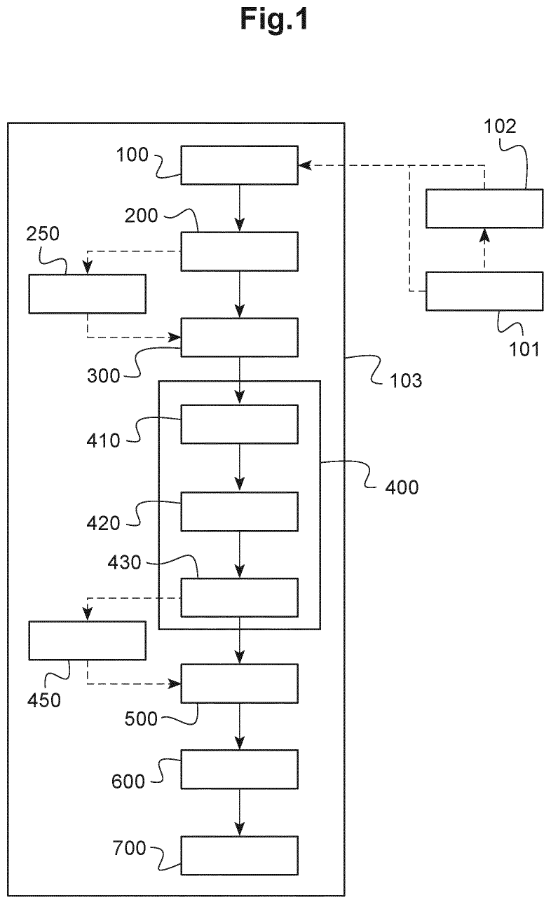

FIG. 1 is a block schematic of a method according to the invention,

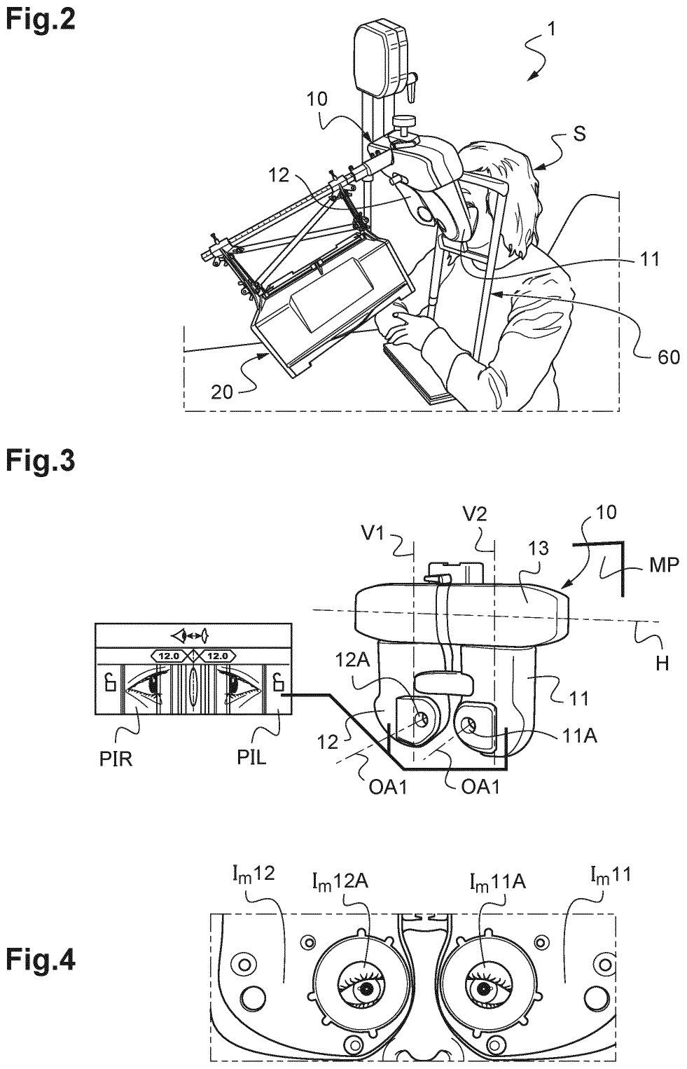

FIG. 2 is a schematic view of an optometry device used to implement said method according to the invention,

FIG. 3 is a schematic view of the refraction test unit of the optometry device of FIG. 2, with two side pictures of the subject used to determine the vertex distances,

FIG. 4 is a schematic view of an image captured by a camera to check the centering of the eye with the refraction test unit,

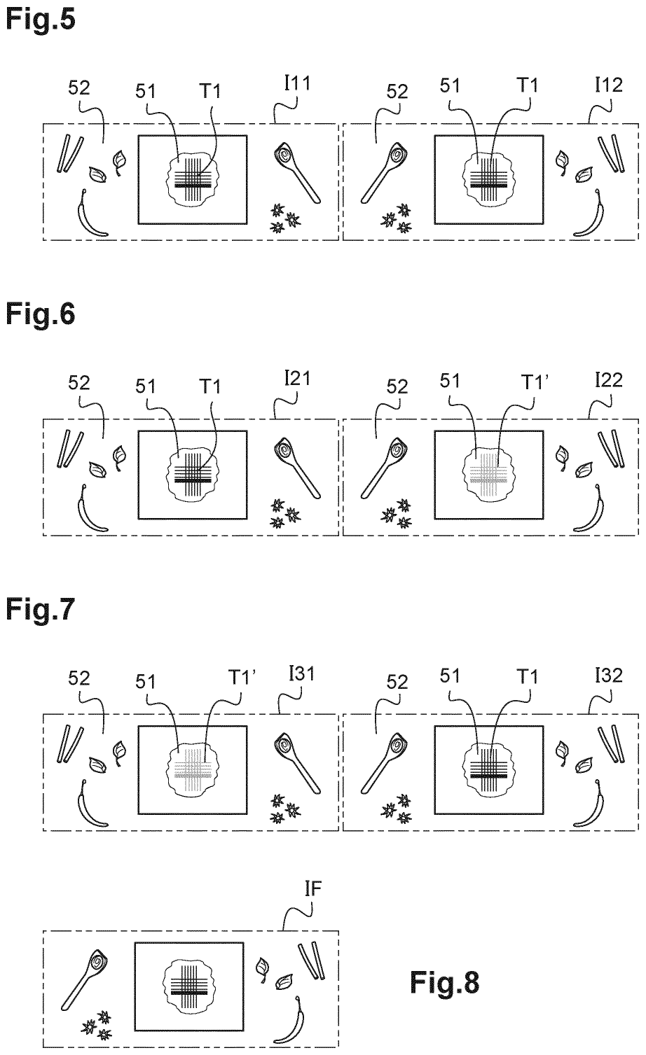

FIG. 5 shows an example of the first and second test images displayed by the optometry device of FIG. 2 during implementation of the method of FIG. 1, while measuring a value of spherical refraction of both eyes of the subject,

FIGS. 6 and 7 show examples of the first and second test images displayed by the optometry device of FIG. 2 during implementation of the method of FIG. 1, while measuring a value of spherical refraction of one eye of the subject (FIG. 6) and while measuring a value of spherical refraction of the other eye of the subject (FIG. 7);

FIG. 8 is a schematic view of the image seen by the subject while the images of FIG. 5, 6 or 7 are displayed,

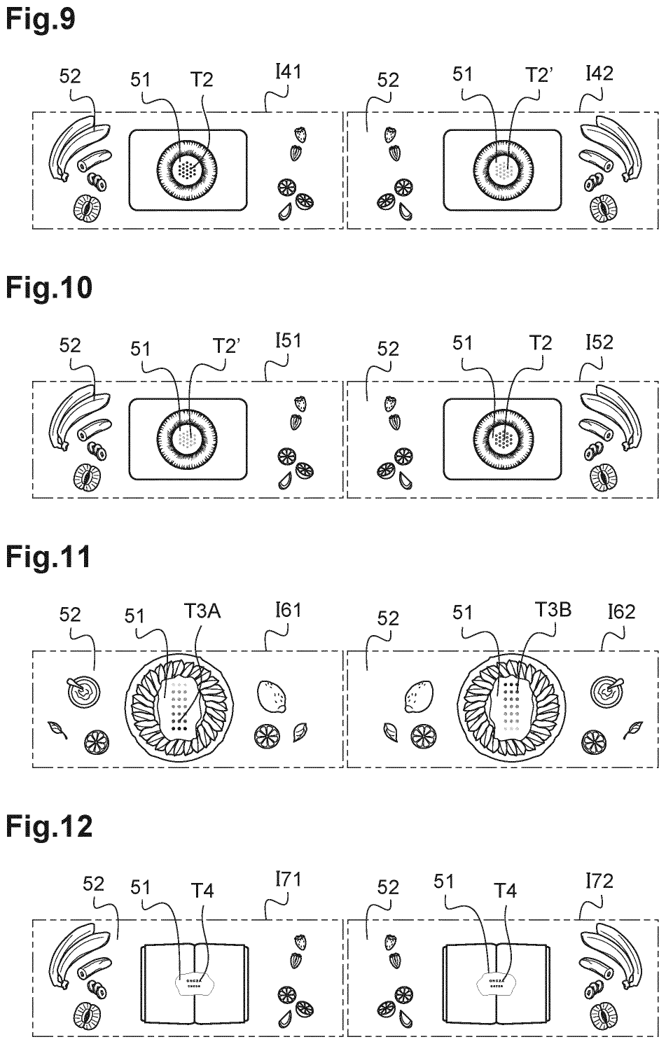

FIGS. 9 and 10 show examples of the first and second test images displayed by the optometry device of FIG. 2 during implementation of the method of FIG. 1, while measuring a value of cylindrical refraction of one eye of the subject (FIG. 9) and while measuring a value of spherical refraction of the other eye of the subject (FIG. 10);

FIG. 11 shows an example of the first and second test images displayed by the optometry device of FIG. 2 during implementation of the method of FIG. 1, while checking the binocular balance of both eyes of the subject,

FIG. 12 shows an example of the first and second test images displayed by the optometry device of FIG. 2 during implementation of the method of FIG. 1, while checking binocular visual perception of one of the eyes of the subject,

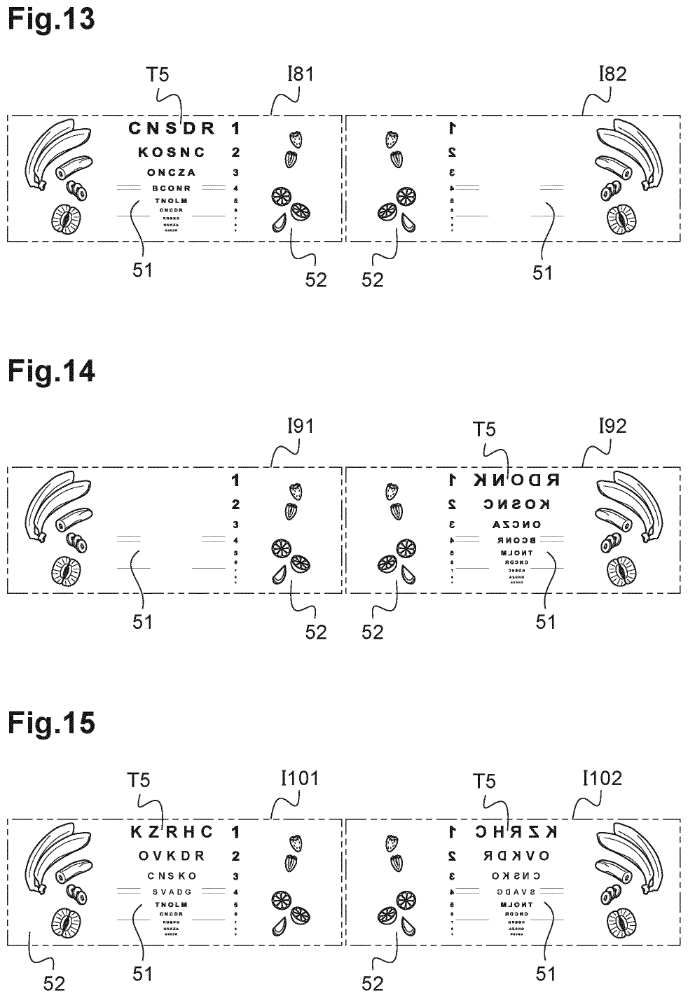

FIGS. 13 and 14 show examples of the first and second test images displayed by the optometry device of FIG. 2 during implementation of the method of FIG. 1, while determining the visual acuity of one eye of the subject (FIG. 13) and while determining the visual acuity of the other eye of the subject (FIG. 14);

FIG. 15 shows an example of the first and second test images displayed by the optometry device of FIG. 2 during implementation of the method of FIG. 1, while determining the visual acuity of both eyes of the subject,

FIG. 16 shows an example of the first and second test images displayed by the optometry device of FIG. 2 during implementation of the method of FIG. 1, while determining the spherical power of both eyes of the subject,

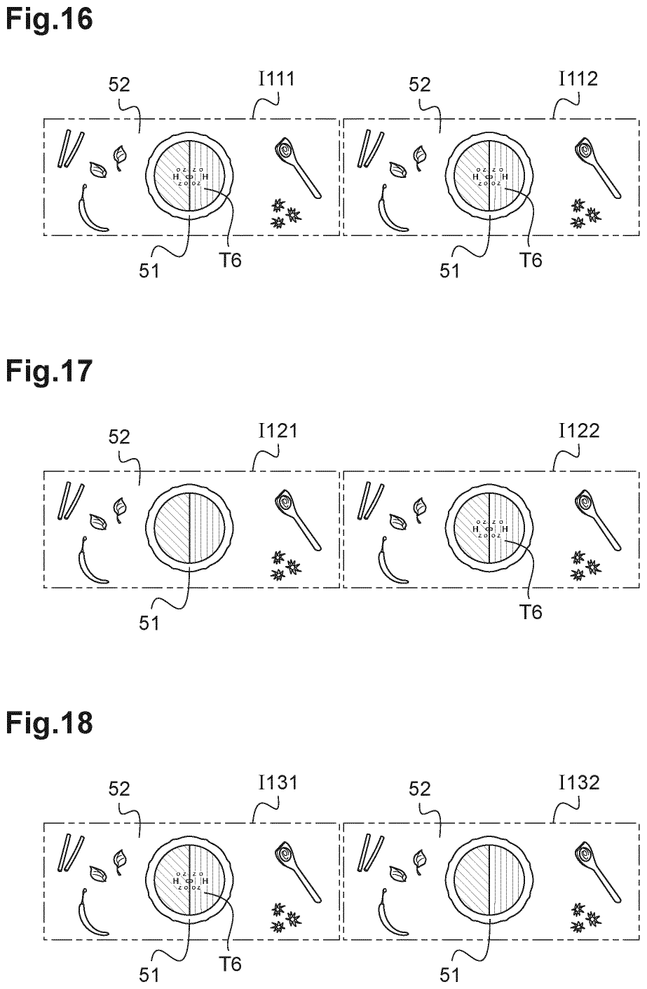

FIGS. 17 and 18 show examples of the first and second test images displayed by the optometry device of FIG. 2 during implementation of the method of FIG. 1, while determining the spherical power of one eye of the subject (FIG. 17) and while determining the spherical power of the other eye of the subject (FIG. 18).

FIG. 1 shows a block diagram of a method for determining a complete set of values of refractive features of an eye of a subject in far and near and/or intermediary vision conditions according to the invention.

This method comprises a determination (block 101 of FIG. 1) of accurate values of refractive features of an eye of a subject in far vision conditions and a determination of accurate values of refractive features of an eye of a subject in near and/or intermediary vision conditions (block 103 of FIG. 1) according to the invention.

Before implementing the method for determining the refractive feature of the eyes of the subject in near and/or intermediary vision conditions, the refractive features of the eye in far vision conditions are preferably determined. This may be done through a method similar to the method used in near or intermediary vision, as described later, or by determining the refraction features of optical equipment currently worn by the subject and adapted to his far vision.

Moreover, in a preferred embodiment, before implementing the method for determining the refractive feature of the eyes of the subject in near and/or intermediary vision conditions, a test (block 102 of FIG. 1) for determining the need for an accurate correction of astigmatism of the eye is performed, comprising measuring the visual acuity of the eye with test images having different contrasts.

If an accurate correction of astigmatism of the eye is needed, the determination of the refractive features of the eye in near or intermediary conditions is especially useful.

The method according to the invention may be performed thanks to an optometry device 1, the main elements of which are represented schematically, in perspective, on FIG. 2.

This optometry device 1 is a binocular device, allowing determination of the refraction feature of each eye of the subject based on a binocular measurement performed while the subject has both eyes E1, E2 opened and un-obstructed.

More precisely, during said binocular measurement, each eye E1, E2 of the subject is provided with a test image. The test images provided to both eyes are configured such that the fusion of the two test images by the brain of the subject may occur. Preferably, the two test images are stereoscopic images providing a representation IF at least partially in three dimensions for the subject, an example of which is shown on FIG. 8.

In order to enable the perception in three dimensions of the two test images by the subject, each test image is configured to be accurately aligned with the corresponding eye of the subject.

The optometry device 1 used in the method according to the invention comprises a binocular refraction test unit 10 with a first optical refraction element 11 adapted to provide different vision correction powers along a first optical axis OA1 and a second optical refraction element 12 adapted to provide different vision correction powers along a second optical axis OA2 (FIGS. 2 and 3).

Said first optical refraction element 11 is configured for providing the first eye of the subject with a first correction power, and said second optical refraction element 12 is configured for providing the second eye of the subject with a second correction power.

By correction power, it is meant a dioptric power allowing correcting a refraction error of the eye of the subject, such as spherical power, cylindrical power and/or axis, prismatic power . . . .

The optometry device 1 also comprises a display system 20 for providing a first and a second test images, the first test image being conveyed to the first optical refraction element 11, and thus to the first eye of the subject, along a first optical pathway and the second test image being conveyed to the second optical refraction element 12, and thus to the second eye of the subject, along a second optical pathway (FIG. 2).

The image display system 20 is thus configured for providing the first test image I11; I21; I31; I41; I51; I61; I71; I81; I91; I101 to the first eye of a subject S and, at the same time, for providing the second test image I12, I22; I32; I42; I52; I62; I72; I82; I92; I102 to the second eye of the subject S, the second test image being different from the first test image.

The first test image I11; I21; I31; I41; I51; I61; I71; I81; I91; I101; I111; I121; I131 is seen by the first eye of the subject S through the first optical refraction element 11, while the second test image I12, I22; I32; I42; I52; I62; I72; I82; I92; I102; I112; I122; I132 is seen by the second eye of the subject S through the second optical refraction element 12.

Each of the first and second optical refraction elements 11, 12 comprises a lens, a mirror or a set of such optical components, that has adjustable refractive power features.

In the example shown on the appended figures, and described in the following, each of said first and second optical refraction elements 11, 12 comprises a lens with variable power. It comprises here a deformable liquid lens having an adjustable shape. The optical axis OA1, OA2 mentioned before thus correspond to the optical axis of the corresponding lens (FIG. 3).

Alternatively, or in addition, the optical refraction element may comprise an ensemble of non-deformable lenses having different optical powers, and a mechanical system that enables to select some of these lenses to group them to form the set of lenses through which the subject can look. In this last case, to adjust the refractive power of the set of lenses, one or several lenses of the set of lenses are replaced by other lenses stored in the refraction test unit. The optical axis mentioned before thus corresponds to the optical axis of the lens placed in front of the eye of the subject.

In particular, each optical refraction element 11, 12 may comprise a lens with variable spherical power and an optical component with a variable cylindrical power and variable cylindrical axis.

Each of the optical refraction elements 11, 12 is intended to be placed in front of one of the eyes of the subject, close to this eye. It is placed, for example, at a distance comprised between 1 and 5 centimeters from the eye.

Each of the eyes of the subject can see said first or second test image displayed by said display system 20 through the lens of the optical refraction element 11, 12, or through the set of lenses, or by reflection onto a mirror of the optical refraction element in an alternative implementation.

In the following, we will describe the optometry device in the case where each optical refraction element 11, 12 comprises an optical set with a lens 11A, 12A with variable spherical power and an optical component with a variable cylindrical power and variable cylindrical axis.

The optical set has an overall spherical power Sph corresponding to the spherical optical power, expressed in diopters. The cylindrical components of its refractive power are those of an equivalent cylindrical lens that has a cylindrical power Cyl (expressed for instance in diopters), and whose cylinder has an orientation represented by an angle A. Each of the first and second refraction correction, provided by the corresponding optical refraction elements 11, 12, may be characterized by the values of these three refractive power parameters Sph, Cyl and A.

This refractive correction could be equally characterized by the values of any other set of parameters representing the above mentioned refractive power features of the optical sets of the optical refraction elements 11, 12, such as the triplet {M, J0, J45}, where the equivalent sphere M is equal to the spherical power S plus half of the cylindrical power C (M=S+C/2), and where J0 and J45 are the refractive powers of two Jackson crossed cylinders lenses representative of the cylindrical refractive features of the lens or of the set of lenses of the optical refraction elements 11, 12.

Said optical refraction elements 11, 12 are mounted on a common support 13 that extends between the optical refraction elements, above them, along a longitudinal axis H (FIG. 3) that is horizontal.

This support 13 is linked to a global supporting structure (not represented or only partially represented on the figures) that lies on a table or on the ground.

Each of the optical refraction elements 11, 12 is mounted on the support 13 to be mobile in rotation about an axis V1, V2 perpendicular to said longitudinal axis H. A mean plane MP of the first and second optical refraction elements 11, 12 goes through these axes V1, V2 (FIG. 3). This mobility of the optical refraction elements allows adjusting the alignment of the optical axes OA1, OA2 with the eyes of the subject taking into account their convergence in near vision conditions. Said display system 20 is adapted to provide said first and second test images, examples of which will be provided hereafter, with details smaller than 1 arc minute, over a field of view of at least 8° in horizontal directions, in near vision conditions.

The distance between the image displayed for each eye and the refraction test unit may be fixed or variable. The variation of the distance between the test images displayed by the display system and the optical refraction elements may be obtained by varying the distance between the display system as a whole and the refraction test unit or by using mobile optical components such as mirrors inside the display system 20.

The test images can be placed at a distance of near vision, 40 centimeters for example, or intermediary vision, 60 cm for example, of the eyes of the subject.

Said binocular refraction test unit 10 is advantageously mobile between at least two test configurations: a horizontal vision test configuration where the first and second optical refraction elements are positioned in a first position where said first and second optical axes extend horizontally and an inclined vision test configuration where the first and second optical refraction elements are inclined compared to said first position, so that said first and second optical axes are inclined downwards.

Preferably, said binocular refraction test unit is mobile as a whole in rotation about a horizontal rotation axis parallel to the mean plane of the first and second optical refraction elements.

As a consequence, during the determination of the refraction features of the eyes of the subject in near/intermediary vision, the optical axes OA1, OA2 of said optical refraction elements 11, 12 are preferably inclined downwards in order for the subject to be placed in an ergonomic near vision position.

Here, as shown on FIG. 2, the display system 20 is housed in a box mounted on a rail. Said rail is attached to the common support 13 for the optical refraction element 11, 12. The rail extends globally perpendicularly to the mean plane of said optical refraction element 11, 12. The display system 20 may then be translated along the rail to change the distance to the eyes of the subject. It may also pivot as a whole with the refraction test unit 10. This rail can be used to test the quality of near and/or intermediate correction. Because the display system 20 can be moved, the accommodation amplitude and the range of sharpness of the target or optotype can be tested.

As the image display system 20 is not the object of the present invention, it will not be described in full details. Any image display system 20 able to provide test images in stereoscopic vision may be used.

In particular, the display system may be held in the hands of the subject.

It may also be placed on a table, for example in an inclined position.

In particular, the display system 20 used in the method according to the invention may comprise one or two screens for displaying said test images.

Example of test images as displayed by said screens are shown on FIGS. 5 to 7 and 9 to 15. One of the two test images (the right image of each figure) is transmitted to the corresponding eye without reflection or with an even number of reflections. The other test image (left image of the figures) is transmitted to the eye with an odd number of reflections. In order for the other test image to be provided to the eye in the appropriate orientation, the screen displays the mirror image of the other test image. FIGS. 16 to 18 show examples of test images as displayed by said screens where the two test images are transmitted to the corresponding eye both with an odd number or even number of reflections.

For example, a liquid-crystal display screen may be able to display the first test image with a first polarization, and, at the same time, to display the second test image with a second polarization. Two screens displaying the first test image with a first polarization and the second test image with a second polarization may also be used. The first and second polarizations are orthogonal to each other. For instance, the first and second polarizations are both rectilinear and perpendicular to each other. Or, similarly, the first polarization is a left-hand circular polarization while the second polarization is a right-hand circular polarization.

The whole extent of the screen or two screens can be seen through each of the first and second optical refraction elements 11, 12.

But the first optical refraction element 11 comprises a first polarizing filter that filters the light coming from the image display system 20. The first polarizing filter filters out the second polarization and lets the first polarization pass through so that it can reach the first eye of the subject. So, through the first optical refraction element 11, the first eye of the subject can see the first test image, but not the second test image.

Similarly, the second optical refraction element 12 comprises a second polarizing filter that filters the light coming from the image display system 20. The second polarizing filter filters out the first polarization and lets the second polarization passes through so that it can reach the second eye of the subject.

The image display system may use any other separation technic, such as «active» separation for which each image test is displayed alternatively at a high frequency while a synchronized electronic shutter or polarizers are blocking the eye for which the image should not be addressed. Separation system could also use chromatic separation with chromatic filters both on the display and the eye in which each side/eye has different chromatic filters that block each other (for example red and green filters).

The optical device used to implement the method according to the invention is controlled by a computer programmed to perform corresponding steps of the method. This computer receives the results of the measurements performed and inputs of the answers of the subject to the subjective visual tests performed during the method, as described hereafter.

In particular, the subject may be provided with a joystick, a mouse, a keypad, or other input devices in order to provide the answers to the subjective tests.

According to the invention, the method 103 for determining the refraction features of the eyes of the subject in near and/or intermediary vision comprises at least the following steps:
a) adjusting 100 the relative position of the subject and said refraction test unit so that the pupils of his eye are aligned with said first and second optical axes in near and/or intermediary vision conditions,
b) determining 200 a vertex distance between said first or second optical refraction element and said first or second eye of the subject in near and/or intermediary vision conditions,
c) determining 300 a value of a parameter representative of an accommodation feature of the eyes of the subject in near and/or intermediary vision conditions,
d) determining 400 preliminary values of said refractive features of the eyes of the subject in near and/or intermediary vision conditions, comprising at least a preliminary value of the spherical refraction of each eye in near and/or intermediary vision conditions and a preliminary value of the cylindrical refraction of each eye in near and/or intermediary vision conditions,
e) checking 500 binocular visual perception of the subject by adding to the vision correction powers provided to the eyes an added predetermined spherical power,
f) determining 600 a final visual acuity of the subject in near and/or intermediary vision conditions, and
g) determining 700 said accurate values of refractive features of an eye of a subject in near and/or intermediary vision based on the results of the previous steps,
wherein, in said step d), the following steps are performed in this order:
   measuring 410 a first value of spherical refraction of each of the eyes of the subject,
   measuring 420 said preliminary value of cylindrical refraction of each of the eyes,
   measuring 430 a second value of spherical refraction of each of the eyes of the subject, said preliminary value of spherical refraction of each of the eyes of the subject being determined based on said second value.

During said steps a) to e), said first and second optical axes of said optical refraction elements are preferably inclined downwards.

An ergonomic near or intermediary vision position is then provided to the subject, making the measures performed more accurate.

The method is implemented by a control unit comprising for example a computer with at least a processor and a memory.

### Step a)

In step a), the relative position of the subject and said refraction test unit 10 is adjusted so that the pupils of the eyes of the subject are aligned with said first and second optical axes OA1, OA2 of the optical refraction elements 11, 12 in near and/or intermediary vision conditions.

In order to achieve that, the position and orientation of the head of the subject may be modified.

The position and orientation of the first and second optical axes OA1, OA2 of the refraction test unit 10 may also be modified.

In particular, the optical refraction elements 11, 12 may be moved in their mean plane, along the longitudinal axis H of the support 13. This allows adjusting the distance between the two optical refraction elements 11, 12 depending on the interpupillary distance of the subject or on the monocular pupillary distances of the subject.

The optical refraction elements 11, 12 may be pivoted around their vertical axes V1, V2 to allow adjusting the alignment of the optical axes OA1, OA2 with the eyes of the subject taking into account their convergence in near vision conditions.

The head of the subject may be guided toward an appropriate position thanks to a head support 60 (FIG. 2) where the subject may rest his chin and/or forefront.

In order to check the alignment and further guide the movements of the optical refraction elements and head of the subject, an alignment verification device comprising a camera oriented towards the eyes of the subject or an eye tracking device may be used.

In particular, the alignment verification device may comprise a retractable camera adapted to be placed in front of the subject, for example in front of the middle of one of the screens, for verifying the alignment and removed when the eyes are tested. The camera, when activated, has indeed preferably an optical axis close to the center of the test image, so as to avoid parallax error when checking eye alignment. When not in use, the camera is retracted out of the optical path of the light emitted by the screens.

The retractable camera is therefore mobile between two positions: a first position where it is placed in front of the head of the subject, with its optical axis perpendicular to the mean plane of the optical refraction elements 11, 12 of the refraction test unit 10, and a second position where it is out of the optical path of light in the optometry device 1.

Alternatively, a fixed camera, placed outside of the optical pathways of the display system may be used.

The eye alignment verification can be done by comparing the position of the center of eye pupils with the center of the refraction test unit optics or the optical axis of each of the optical refraction elements 11, 12. The optical axis or center of the optics and the eye pupil positions can be determined from image processing, by detecting for example round edges of the optics and the pupil circular shape. Alternatively, instead of detecting the round edge of the refraction test unit optics, a known pattern can be used, for example identifying a small printed cross at the center of the lenses of the optical refraction test elements.

The camera is preferably a near Infra-Red camera, so as to easily capture eye features such as pupil position.

An image captured by such a camera is shown on FIG. 4. The image of the pupils of the eyes of the subject S and the edge of the image of the lenses Im11A, Im12A of image the optical refraction elements Im11, Im12 may be identified and their positions compared to check the alignment of the center of the pupils with the center of the lenses 11A, 12A.

The alignment verification device may also comprise an eye tracking device or a sensor. The eye tracking device may be fixed on the refraction test unit 10 near the eyes, for example close to the lenses 11A, 12A. The sensor may be integrated to one of the screens of the display system 20 or added to the display system 20. The data generated by the eye tracking device or sensor may give indications from which the orientation of the eyes and gaze directions may be deduced. Data from the sensor may also be used to determine the distance between the eye and the corresponding test image.

The alignment verification device may also comprise a sensor attached to the refraction test unit for measuring the inclination of the refraction test unit.

Alternatively, using an eye tracking device, the gaze directions of the eyes of the subject may be measured and compared to the directions of the optical axes OA1, OA2 to check that they are parallel and preferably overlaid.

Finally, the camera may also be used to determine near vision distance. Elements of the refraction test unit having a known size (printings, edge of the lens of the optical refraction elements . . . ) may be identified on the image captured by the camera. The distance from the camera to the refraction test unit 10 may then be derived from the pixel size of the images of these elements.

When using an eye tracking device, the eye behavior may be determined. The dynamic tracking may synchronize with screens. Thanks to the eye tracking device, the position of the head of the subject, the position of the eyes, the gaze direction and distance from the eyes to the target used in different visual activities (reading a book, using a smartphone, using a computer, reading music), can be determined and may be taken into account while performing the steps of the method according to the invention and determining the refraction features of the eyes of the subject.

In a variant, the so-called Harmon distance for the subject and/or the natural posture of the subject may be determined and taken into account when determining the refraction features of the eyes of the subject. The Harmon distance is the appropriate working distance of the subject for near activities. Any device allowing measuring the working distance of the subject in near and/or intermediary vision conditions can be used.

This step a) ensures that the eyes are precisely aligned with the test images displayed by the display system 20. The stereoscopic peripheral components of said test images can then be seen in three dimensions by the eyes. The comfort of the subject and the accuracy of the refraction values of the eyes measured are increased.

Step a) is preferably performed before performing the other steps of the method according to the invention. The other steps of the method according to the invention that require measurements of visual performance or features of the subject are performed while the relative position of the subject and said refraction test unit is adjusted according to this step a). In particular, steps b) to f) are performed while the relative position of the subject and said refraction test unit is adjusted according to this step a).

### Step b)

In step b), at least a profile image of the subject S placed in front of the refraction test unit is acquired.

Preferably, two profile images PIR, PIL of the subject, each taken from a different side of the subject, are acquired.

These profile images are for example captured thanks to Infra-Red cameras housed inside the refraction test unit 10 and oriented towards the eyes.

Examples of such profile images PIR, PIL are shown on FIG. 3.

Each profile image PIR, PIL shows an image of one of the eyes of the subject and the face of the lens placed close to it.

The vertex distance between each eye of the subject and the corresponding optical refraction test element is then deduced from this profile image.

This vertex distance may be deduced from the distance measured on said profile image PIR, PIL between the apex of the image of the eye and the apex of the image of the face of the corresponding lens.

In the case where a single profile image is captured, the vertex distance of each eye is determined to be the same based on the single profile image.

In the case where two profile images are captured, a vertex distance is determined for each eye.

The vertex distance is for example comprised between 11 and 13 millimeters in far vision. It is for example comprised between 17 and 19 millimeters in near vision.

It depends on the morphology of the head of the subject and on the relative position of the head and optometry device. It also depends on the position (horizontal or inclined) of the optical axes OA1, OA2 of the optometry device.

The vertex distance between each eye and the corresponding optical refraction element 11, 12 during the test being determined, it can be taken into account in step g) for determining said accurate values of refractive features of the eye of the subject in near and/or intermediary vision.

This vertex distance may also be compared to the distance between the eye and the lens of the eyeglasses prepared for the subject. The value of corrective powers of the ophthalmic lenses of the eyeglasses may be determined by taking into account said vertex distance value measured in step b) and the distance between the eye and the lens of the eyeglasses prepared for the subject, and in particular their difference.

Alternatively, the vertex distance is not measured but predetermined by positioning the head of the subject such that the vertex distance is equal to this predetermined value, for example comprised between 10 and 40 millimeters, for example equal to 25 millimeters.

The vertex distance is preferably measured for each eye while the subject and the refraction test unit are in the adjusted relative position determined in step a).

In method according to the present disclosure, the vertex distance is kept constant for all the subsequent steps carried out using the refraction test unit 10.

### Optional Step of Preliminary Visual Acuity Test

Optionally, before step d) or, preferably, between steps b) and c), a preliminary visual acuity test 250 (FIG. 1) of the eyes of the subject is performed.

This step is performed with near or intermediary vision conditions corresponding to the vision conditions of the test currently performed.

Initial values of optical powers, in particular initial spherical power and/or cylindrical power and axis are applied to the optical set of each optical refraction element 11, 12, here to the variable power lenses 11A, 12A and the optical component with a variable cylindrical power and variable cylindrical axis.

These initial values of optical powers may be determined based on refraction features of a current optical equipment of the subject, for example the optical powers of the ophthalmic lenses of current eyeglasses of the subject.

They may be determined based on previous values of refraction of the eyes determined during a previous test of the subject. This previous test may have been performed at an earlier date, for example while testing the subject's vision to prescribe him current or previous optical equipment.

These initial values of optical powers may also be determined based on the refraction features of the eyes of the subject measured in far vision conditions immediately before performing the method of the invention or preferably on the same day, within the last 24 hours before.

The preliminary visual acuity test 250 is performed by testing each eye separately from the other, and/or by testing both eyes simultaneously.

Each tested eye is provided with a test image comprising a visual target. In the case where only one of the two eyes is tested, the other eye is provided with an image with no visual target.

The visual target comprises for example a column of optotypes such as letters of different sizes. It is similar to the target T5 of the test images of FIGS. 13 to 15, with only one letter per line.

The question asked to the subject may be “In the column of letters that I present to you, what is the smallest line of letters that you can just read, without forcing, without squinting, without trying to get closer? You can find your way by giving me the number of the corresponding line”.

The determination of this preliminary visual acuity gives information relative to the necessity of performing the further steps of the method and also on the difference between the initial values of optical powers used and the refraction features of the eyes of the subject in near vision.

Preferably, the preliminary visual acuity test is performed while the subject and the refraction test unit are in the adjusted relative position determined in step a) and with the vertex distance of each eye determined in step b).

### Step c)

In a following step c), a value of a parameter representative of an accommodation feature of the eyes of the subject in near and/or intermediary vision conditions is determined. This step c) is performed for each eye, while maintaining the vertex distance determined at step b) and at the relative position of the subject and refraction test unit determined at step a).

Preferably, said parameter representative of an accommodation feature of the eyes of the subject in near and/or intermediary vision conditions is a binocular parameter.

A binocular accommodation test is performed to determine said value of a parameter representative of an accommodation feature of the eyes by presenting each of the eyes of the subject with an identical target placed at near or intermediary vision optical distances from the eyes of the subject and modifying the spherical powers of the lens of each of the first and second optical refraction elements to determine the minimum spherical powers for which the subject accommodates on the target. Said identical targets are presented to both eyes simultaneously.

Each of said identical targets comprises for example several lines of optotypes. The initial values of the spherical power of the optical refraction elements 11, 12 is for example determined based on refraction features of a current optical equipment of the subject, for example the optical powers of the ophthalmic lenses of current eyeglasses of the subject, on previous values of refraction of the eyes determined during a previous test of the subject, or on the refraction features of the eyes of the subject measured in far vision conditions before performing the steps of the current method. No cylindrical power is used.

The spherical power of the optical refraction elements 11, 12 is progressively increased until the subject indicates that he can distinguish the optotypes.

An accommodation range of the subject may then be determined. The accommodation range is relative to the distance between the furthest point that can be seen sharply when the eye is relaxed, i.e. without accommodation and the closest point that can be sharply seen when the eye is accommodating.

The accommodation range may also be expressed as a spherical refraction range of the subject, quantifying the range between the spherical refraction of the eye of the subject when it accommodates on the closest point that can be seen sharply when the eye is accommodating and the furthest point that can be seen sharply when the eye is relaxed.

In another embodiment, the parameter representative of the accommodation may refer to the spherical refraction of at least one of the eyes in a specific accommodation state, for example at a maximum accommodation in near vision or when relaxed in far vision.

According to this embodiment, visual tests are carried out with the refraction test unit 10 to determine the spherical refraction of each eye in far vision, without correction. Preferably, these visual tests are similar to the test explained above. These visual tests provide a value of the spherical power of the set of optical refraction elements 11, 12.

Based on the value of the parameter representative of the accommodation of the eye of the subject, the spherical refraction of each eye in a relaxed state may be determined. It is stored in the memory of the computer and used as a starting point for step d) of the method according to the present disclosure.

### Step d)

After the steps already described, preliminary values of said refractive features of the eyes of the subject in near and/or intermediary vision conditions are determined in said step d). For example, near and/or intermediary vision conditions refer to visual tests that are performed at a fixed distance from the eyes of the subject. The fixed distance is comprised into a range of distances between 20 to 60 centimeters. According to a preferred embodiment, the visual tests are performed at a fixed distance of 40 centimeters from the eyes of the subject. The vertex distance and the relative position determined in steps a) and b) are maintained during step d).

More precisely, in step d), at least a preliminary value of the spherical refraction of each eye in near and/or intermediary vision conditions and a preliminary value of the cylindrical refraction of each eye in near and/or intermediary vision conditions are determined.

In practice, in said step d), the following steps are performed in this order:
d1) measuring a first value of spherical refraction of each of the eyes of the subject,
d2) measuring said preliminary value of cylindrical refraction of each of the eyes,
d3) measuring a second value of spherical refraction of each of the eyes of the subject, while taking into account the preliminary value of cylindrical refraction, said preliminary value of spherical refraction of each of the eyes of the subject being determined based on said second value.

Preferably, for the following steps of the method according to the present disclosure, the preliminary value of the spherical refraction of the eye of the subject is equal to the second value of the spherical refraction determined in step d3).

In the following paragraphs, the preliminary value of cylindrical refraction refers to astigmatism features of each eye of the subject and thus comprises the cylinder and/or the axis of each eye.

Steps d1) and d2) allow searching for the spherical and cylindrical refraction values of the eye, while step d3) allows refining the spherical refraction value of the eye.

The first value of spherical refraction is determined without changing any cylindrical refraction value of the eyes. It is done through a classic subjective test, by presenting targets or optotypes to the subject and asking him to assess the quality of his perception of the targets or optotypes. The subject is usually asked to compare his perception of two different targets or optotypes.

For example, in step d1), the initial value of the sphere power of the set of optical refraction elements 11, 12 used in step d1) corresponds to the accommodation feature determined in the step c). Then, the initial sphere power of the set of optical refraction elements 11, 12 is changed to determine the first value of the spherical refraction of the eye in near and/or intermediary vision conditions, for example through classical subjective refraction tests.

Only the value of the spherical power of the set of the optical refraction elements 11, 12 is changed to determine the first value of spherical refraction of each eye of the subject is measured. No cylinder power is provided by the optical refraction elements 11, 12 in step d1) and no cylindrical refraction of the eye is determined.

Step d1) allows determining a first value of the spherical refraction of each eye of the subject.

Then, after the determination of this first value of spherical refraction of each eye, astigmatism features comprising the cylinder and/or the axis of each eye of the subject are determined in step d2).

Preferably, step d2) is performed while keeping the equivalent sphere of the set of the optical refraction elements 11, 12 constant and equal to the equivalent sphere provided by the first value of the spherical refraction determined in step d1). It means that during step d2), the value of the spherical power of the set of refraction elements 11, 12 is modified when the cylindrical power and/or the axis of the refraction element 11, 12 is modified in order to maintain the equivalent sphere constant.

The cylinder and/or the axis of each eye determined in step d2) correspond to the preliminary value of cylindrical refraction.

Then, when the cylinder and/or the axis of each eye are determined, the second value of spherical refraction of each eye is determined in step d3).

In step d3), the value of the cylindrical power of the set of optical refraction elements 11, 12 is kept constant, equal to the preliminary value determined in step d2). Step d3) allows refining again the value of the spherical refraction of each eye determined in step d1) by taking into account the astigmatism features of the eyes.

The second value of the spherical refraction is then determined while providing said preliminary value of cylindrical refraction to the eye. This second value corresponds to the preliminary value of the spherical refraction.

The subjective test may comprise the comparison of target or optotypes simultaneously displayed on backgrounds of different colors, such as green and red or the comparison of the perception of optotypes of different sizes displayed one after the other, and/or the comparison of the perception of different part of the target and/or the comparison of the perceptions of the same targets/optotypes with different spherical power or cylindrical power and axis or cylindrical axis of the lenses 11A, 12A of the optical refraction elements 11, 12.

For example, step d1) may be performed with the test images I11, I12, I21, I22, I31, I32 represented schematically on FIGS. 5 to 7, where the target T1 is a cross. The question asked to the subject may be: “Here is a cross, do the lines seem blacker, sharper, more contrasted horizontally, vertically or are they equally contrasted?” Positive increment in spherical power is added for each tested eye until the subject says he sees the horizontal and vertical lines equally black.

Alternatively, step d1) may also be carried out with a red/green duo-chrome test, as described in reference to the step D1) in far vision.

Test images I111, I112, I121, I122, I131, I132 used in such a red/green duo-chrome test are for example shown on FIGS. 16, 17 and 18. FIG. 16 shows the images used in a binocular duochrome test of both eyes at the same time, whereas FIGS. 17 and 18 show the test images shown when testing one eye separately from the other, thereby testing only one eye while keeping the other eye opened and unobstructed.

On these figures, the tested-eye target T6 of the tested-eye test images I122, I131 comprises a line of optotypes. The central part 51 of each test image I111, I112, I121, I122, I131, I132 comprises a red half and a green half represented by different hatching patterns. The tested-eye target T6 is then more precisely a line of letters on a red-green background. The non-tested-eye test images I121, I132 comprises no target in the red and green central part. When testing both eyes at the same time (FIG. 16), both test images comprise the red/green central part with a tested-eye target T6.

A search for the maximum convex sphere is carried out with such a red green R/V duo-chrome test. Its goal is to find the sphere which gives the best sharpness (objective −0.1 Log MAR). The subject must choose whether he sees the letters blacker on one of the two backgrounds, or if the contrasts of the letters are the same. If possible, this test is performed with a 0 LogMAR acuity line, if the subject can read the entire line of letters, otherwise on a lower acuity line.

The question asked to the subject may be: “Tell me if you find the letters more contrasting on red, green, or if you find them identical”

The preliminary value of cylindrical refraction a preliminary value of cylindrical refraction is determined by any known protocol.

The preliminary values of the spherical and cylindrical refraction values of the eye, may comprise a spherical power Se (expressed for instance in diopters) and a cylindrical power Ce (expressed for instance in diopters) having an orientation represented by an angle Ae. The preliminary values of the spherical and cylindrical refraction values of the eye may also comprise the values of any other set of parameters representing the above mentioned refractive power features of the eyes, such as a triplet {Me, J0e, J45e}, comprising the equivalent sphere Me equal to the spherical power Se plus half of the cylindrical power Ce (Me=Se+Ce/2), and where J0e and J45e are the refractive powers of two Jackson crossed cylinders lenses representative of the cylindrical refractive features of the eye.

For example, the step of determining the preliminary value of cylindrical refraction is performed with a cross cylinder test. The cross cylinder comprises a test lens with a spherocylindrical combination of +0.25 diopter in spherical power and −0.50 or −0.33 diopter in cylindrical power.

No matter what set of parameters is used to describe the refractive power of the eye, as mentioned above said step d2) is performed in such a way that the equivalent sphere of the optical set of the optical refraction elements 11, 12 remains constant while this step is performed. In other words, during step d2), the cylinder power and axis or the refractive powers J0, J45 of the optical sets of the optical refraction elements 11, 12 are modified to test the eye, and the spherical power Sph of the optical sets may also be modified to ensure that the equivalent sphere remains constant.

In practice, when a change of cylinder power or refractive power J0, J45 over 0.5 D is made in step d2), the value of the spherical power of the optical set of the optical refraction elements 11, 12 is also modified to keep the equivalent sphere constant.

The final values obtained for the cylindrical power and axis or for the refractive power J0, J45 of the optical refraction elements 11, 12 in step d2) are taken into account as being the preliminary values of cylindrical refraction of the eye sought in step d2).

The test images I41, I42, I51, I52 used for this step d2) comprise a target T2 with a cloud of black dots on a green background, as shown for example on FIGS. 9 and 10. The subjective test comprises comparing two positions of the cross cylinder by simply turning the test lens over and then asking in which position the subject finds the image sharper, or more uniform.

The question asked to the subject may be: “I am going to compare two positions, you will tell me if you perceive the points sharper, more contrasted in one of the two positions, or if they seem identical”. It is also possible to use the letters to test in which position these letters are sharp, more contrasted or if they seem identical in both positions.

In step d3), the second value of spherical refraction is determined while taking into account the preliminary value of cylindrical refraction of the eyes determined in step d2). In practice, the optical sets of the optical refraction elements 11, 12 of the refraction test unit 10 provide the preliminary value of cylindrical refraction determined in step d2) while the second spherical refraction values of the eyes are determined, leading to more precise spherical refraction values. This step d3) is performed through any classic subjective test as it was the case for step d1).

Preferably, each of steps d1), d2) and d3) is performed with binocular vision of the subject. Two test images are provided to the eyes of the subject: one test image is provided to each eye of the subject. Both eyes remain open and unobstructed. Each eye therefore sees the corresponding test image.

Each of these steps d1), d2) and d3) may be performed by testing both eyes simultaneously in binocular vision, or by testing each eye separately from the other while maintaining a binocular vision.

As described in more detail hereafter in reference to some examples of implementation, while testing both eyes simultaneously in binocular vision, the two test images provided to the eyes of the subject both comprise a test target or optotype. While testing only one of the eyes of the subject, the test image provided to the eye test comprises a test target or optotype, while the other test image has either no target nor optotype or the same target or optotype displayed with lower contrast.

As a consequence, any of the following alternatives is possible:
said first and second values of spherical refraction of each of the eyes of the subject are determined by testing both eyes simultaneously in binocular vision; or
said first value of spherical refraction of each of the eyes of the subject is determined by testing both eyes simultaneously in binocular vision and said second value of spherical refraction of each of the eyes of the subject is determined by testing each eye separately from the other while maintaining a binocular vision; or
said first value of spherical refraction of each of the eyes of the subject is determined by testing each eye separately from the other while maintaining a binocular vision and said second value of spherical refraction of each of the eyes of the subject is determined by testing both eyes simultaneously in binocular vision; or
said first and second values of spherical refraction of each of the eyes of the subject are determined by testing each eye separately from the other while maintaining a binocular vision.

This way, the accommodative state of each eye during the corresponding steps of the method of the invention is close to the accommodative state of the eye during usual visual tasks. Any difference in accommodative state between the two eyes may be taken into account.

Preferably, for determining said preliminary value of cylindrical refraction of each of the eyes, each of the eyes is tested separately from the other while maintaining a binocular vision. This way, the visual state of the eyes during the steps of the method of the invention is close to the visual state of the eyes during the actual use of the corrective lenses.

According to an advantageous embodiment, said first and second values of spherical refraction and said preliminary value of cylindrical refraction of each of the eyes of the subject are determined by testing each eye separately from the other while maintaining a binocular vision and said first values of spherical refraction, said preliminary value of cylindrical power and axis and said second value of spherical refraction are obtained by alternating measures on the first and second eyes of the subject. This way, it is easy to indicate to the subject what is expected from him when the test is performed on the first eye, and then ask him to do the same with the second eye. The steps are then performed in a quick and efficient way.

According to another advantageous embodiment, said first and second values of spherical refraction and said preliminary value of cylindrical refraction of each of the eyes of the subject are determined by testing each eye separately from the other while maintaining a binocular vision and said first values of spherical refraction, said preliminary value of cylindrical power and axis and said second value of spherical refraction are obtained by performing measures exclusively on the first eye and then exclusively on the second eye of the subject.

Said first value of spherical refraction, said preliminary value of cylindrical power and axis and said second value of spherical refraction are then successively obtained for the first eye. They are obtained successively afterwards for the second eye. The steps are then performed in a quick and efficient way as it is not necessary to switch from one eye to the other at each step.

As explained later in more details, some subjects have a binocular vision that is strongly dominated by the image perceived by one of their eyes, called the dominant eye. In this case, for any embodiment, in a general manner it is preferred to start with the non dominant eye of the subject and end with the dominant eye.

For example, when alternating measures on the first and second eye at each step, the first eye is preferably the non dominant eye and the second is preferably the dominant eye at each step.

When the preliminary values are all determined for the first eye and then for the second eye, the first eye is also preferably the non dominant eye and the second one is preferably the dominant eye.

Finally, the preliminary value for the spherical refraction of each eye of the subject is determined based on the second value. The second value is indeed more accurate as it was determined with a correction of the astigmatism of the subject, thanks to the preliminary value of cylindrical refraction determined in step d2). The preliminary value of the spherical refraction is for example equal to the second value.

Alternatively, the preliminary value for the spherical refraction of each eye of the subject may be determined taking into account the age of the presbyopic subjects, using for example the database available at https://www.emconsulte.com/em/SFO/rapport/file_100013.html.

### Optional Step of Adjusting the Binocular Balance

Between steps d) and e), a step of adjusting 450 (FIG. 1) the binocular balance of the eyes of the subject to obtain adjusted values of said refractive features of the eyes in near or intermediary vision conditions is performed.

The binocular balance test may be performed by any known protocol.

The initial value of spherical power and cylindrical power and axis of the lenses of the optical refraction elements 11, 12 are here equal to the preliminary values of refraction of the eyes determined in step d).

These preliminary values are modified by adding or subtracting spherical power of the lenses in order to achieve the binocular balance of the eyes of the subject.

In order to achieve this binocular balance, the preliminary values of spherical are modified to ensure accurate perception of the optotype, for example to keep the visual acuity over a predetermined threshold, while limiting the difference in spherical powers between the two eyes to 0.32 diopter.

The binocular balance is the difference between equivalent additions of the right and left eyes

The equivalent addition of each eye is equal to the equivalent sphere in near vision minus the equivalent sphere in far vision of the corresponding eye.

The step of binocular balance is achieved with both eyes being provided simultaneously with specific targets T3A, T3B, as shown for example on FIG. 11.

Each specific target T3A, T3B comprises several lines of points on green background. The lines of points appear darker from top to bottom on one of the test images and from bottom to top on the other test image.

The vision of the subject is fogged by adding a positive spherical power increment of for example 0.5 diopter, so that the subject is still able to see the lines of points or dots presented for the performance of the test. Each eye sees half of the lines of points or dots darker, and together the two eyes see the middle line.

In the case of an imbalance of the two eyes, the subject will see the top or bottom points darker. Positive spherical power increment is then added on the eye seeing the corresponding test image having the top or bottom darker horizontal lines of points until all the lines of points are seen with uniform darkness.

The question asked to the subject during this test may be: “I'll change the glasses, tell me when the matrix of points is evenly black.”

An adjusted spherical value of the refraction of the corresponding eye is then determined by adding the positive spherical power increment to the preliminary value determined in step d).

When this step is performed, the adjusted values of spherical refraction of the eyes are used in the following steps. In the following step e), said binocular visual perception of the subject is then checked by providing him vision correction powers equal to said adjusted values with an added predetermined spherical power, as described below.

Otherwise, the following steps are performed on the basis of the preliminary values determined in step d).

### Step e)

Step e) of the method according to the present disclosure aims to verify if the preliminary values of spherical refraction and cylindrical refraction of each eye determined in step d) are adapted to the vision of the subject.

In step e), the binocular visual perception of the subject is checked by adding to the vision correction powers provided to the eyes according to the results of step d) an added predetermined spherical power. The cylindrical power determined in step d) is maintained while adding the predetermined spherical power in step e).

In practice, the spherical power of the lenses placed in front of the eyes before performing step e) is either equal to the preliminary value of spherical refraction of the eyes determined in step d), or to the adjusted value of spherical refraction of the eye determined in the optional step of binocular balance.

The test images provided to the eyes of the subject are for example shown on FIG. 12. Both eyes are tested together with binocular vision.

The test image I71, I72 provided to the tested eye comprises a target T4, here in the form of visual acuity lines of optotypes (FIG. 12).

The question asked to the subject may be: “Do you find that the letters are sharper, more contrasting, more blurry in position 1, 2 or are they the same?”

The binocular visual perception of the subject is tested by adding a predetermined value of spherical power to the preliminary value of spherical refraction of the eyes determined in step d), or to the adjusted value of spherical refraction of the eye determined in the optional step of binocular balance and by displaying two test images with targets or optotypes.

The predetermined added value of spherical power may be positive or negative. It is for example comprised between 0.1 and 0.3 diopter or −0.3 and −0.1 diopter, for example equal to 0.25 or −0.25 diopter. The subject is then asked to compare his vision of the target or optotype with and without the predetermined added value.

It is then expected that the vision of the subject is degraded by adding said predetermined added value, indicating that the preliminary or adjusted value is an optimum value. If the visual acuity or the subjective comfort of the subject is indeed lower with addition of the predetermined added value of spherical power, the predetermined added value of spherical power is removed and the preliminary or adjusted spherical refraction value remains unchanged.

If it is not the case, a corrected spherical refraction value of the eyes is determined.

The corrected spherical refraction value of each eye may be equal to the sum of the predetermined added value of spherical power and the preliminary value of spherical refraction of the eyes determined in step d).

Alternatively, when the optional step of binocular balance has been performed, the corrected spherical refraction value of each eye is equal to the sum of the predetermined added value of spherical power and the adjusted value of spherical refraction of the eye determined in the optional step of binocular balance. Step e) is repeated.

A predetermined added value of negative spherical power may also be added. If the gain in visual acuity is significant, for example, a gain of more than 3 letters, then this predetermined added value of negative spherical power is taken into account to determine a new corrected spherical refraction value.

The new corrected spherical refraction value is equal to the sum of the predetermined added value of spherical power and the preliminary, adjusted or corrected value of spherical refraction of the eyes. If the subject does not feel a significant improvement in perception, the spherical refraction value is not changed.

### Step f)

In said step f), a final visual acuity of the subject in near and/or intermediary vision conditions is determined when the eyes of the subject are provided with vision correction powers equal to the preliminary values of spherical and cylindrical refraction of the eyes, or equal to the adjusted value of spherical refraction and preliminary value of cylindrical refraction of the eyes when the binocular balance step has been performed. It is then checked that the visual acuity of the subject is over a predetermined threshold value such as 10 tenth or better for example 0.0 LogMAR of visual acuity.

The visual acuity is determined in binocular vision, for both eyes simultaneously or for each eye separately from the other.

An example of the test images I81, I82, I91, I92, I101, I102 used are for example shown on FIGS. 13 to 15. The test images I81, I92, I101, I102 provided to tested eyes comprise a target T in their center. The target T comprises lines of optotypes of different sizes.

If it is not the case, steps a) to e) are repeated.

In an embodiment, the visual comfort and the reading speed of the subject are also determined for each eye or both eyes together.

According to this embodiment, a visual correction is provided to the eyes of the subject on the basis of the results provided in step f). Then, the reading speed may be checked according to the method disclosed in document EP3622343. Alternatively or additionally, the visual comfort may be checked according to the method disclosed in document EP3622343. If the reading speed or the visual comfort are below a predetermined threshold of reading speed, respectively of visual comfort, steps a) to f) are repeated.

### Step g)

In said step g), said accurate values of refractive features of at least one eye of the subject in near and/or intermediary vision are determined based on the results of the previous steps.

Preferably, the accurate values of refractive features of both eyes of the subject in near and/or intermediary vision are determined in step g).

In particular, said accurate value is determined based on said preliminary or adjusted values of the spherical refraction of the eye and said preliminary value of the cylindrical refraction of the eye, taking into account the vertex distance determined in step b). Said accurate values are equal to said corrected values obtained in step f), that is the corrected values determined in the last step e) performed when step e) is repeated after step f).

Said corrected values of said refractive features of the eyes of the subject determined in step e) are determined in said relative position determined in step a), with the vertex distance determined in step b). They take into account the accommodation parameter determined in step c) and used as a starting point un step d). They also take into account the preliminary values determined in step d), and, optionally, the adjusted values determined in said step of adjusting the binocular balance of the eyes when it is performed. Finally, it takes into account said binocular visual perception checked in step e) and said final visual acuity check performed in step f).

It is important to take into account the vertex distance to obtain accurate values of spherical and cylindrical refraction insofar as the distance between the eye and the corrective ophthalmic lens of the eyeglasses prepared for the subject on the basis of these values may be different from the vertex distance determined in step b).

The distance between the eye and the corrective ophthalmic lens of the eyeglasses prepared for the subject depends on the morphology of the head of the subject as well as the shape of the eyeglasses frame chosen.

Said accurate values of the refractive features of the eye and said distance between the eye and the corrective ophthalmic lens of the eyeglasses prepared for the subject are taken into account to determine accurate values of corrective powers of the ophthalmic lens to be mounted in the eyeglasses intended to be worn by the subject.

During the implementation of the method of the invention, while determining the refraction features of the eyes in near vision, far vision or intermediate vision, the distance between the eye and the corrective ophthalmic lens of the eyeglasses is taken into account. This is useful for designing lenses and accurate fitting of lenses into the corresponding frames, especially for aspheric lenses and customized progressive lenses.

In practice, in the specific example of implementation detailed here, said first and second test images provided by said display system 20 to said first and second eyes of the subject comprise a plurality of peripheral image components displayed to be seen in three dimensions.

The peripheral image components are arranged in a stereoscopic manner on the two test images, such that they appear in three dimensions to the subject when the two test images are seen by the corresponding eyes.

Said test images, or at least their peripheral parts, show realistic activities usually carried out in near vision, such as cooking, reading or using a computer. In this last case, the peripheral part of the test images could comprise a joystick, a mouse, a keypad, or any other usual equipment of the computer.

The activities may be customized for the subject, designed to attract his attention, for example depending on age. Test images of games may be used for kids.

As presented in detail below, each of the first and second test images to be displayed comprises:
a central part 51 for displaying optotypes T4, T5, T6 (FIGS. 12 to 18) or any visual targets T1, T1′, T2, T2′, T3A, T3B or for remaining blank, and
a peripheral part 52 that surrounds the central part 51 and contributes to a well balanced fusion process between the left and right visual pathway, for the subject S. This provides a comfortable binocular vision to the subject.

In each step where each of the eyes is tested separately from the other while maintaining a binocular vision:
an tested-eye test image I21, I32, I41, I52, I81, I92, I122, I131 is provided to the tested eye, comprising images of realistic activities carried out in near vision having a plurality of peripheral image components and a tested-eye target T1, T2, T4, T5, T6 displayed in the center of the test image (FIGS. 6, 7, and 9, 10, 13, 14, 17, 18), and
a non-tested-eye test image I22, I31, I42, I51, I82, I91, I121, I132 is provided to the other eye comprising similar images of realistic activities carried out in near vision having a plurality of peripheral image components and either:
   no target displayed in the center of the non-tested-eye test image (FIGS. 13, 14, 17 and 18) or
   a non-tested-eye target T1′, T2′ displayed with a contrast lower than the contrast of the tested-eye target displayed on the corresponding tested-eye test image (FIGS. 6, 7, 9, 10).

In each step where both eyes are tested simultaneously in binocular vision, a test image I11, I12, I61, I62, I71, I72, I101, I102, I111, I112 is provided to each eye, comprising images of realistic activities carried out in near vision having a plurality of peripheral image components and a test target T1, T2, T3A, T3B, T4, T5 displayed in the center of the test image (FIGS. 5, 11, 12 and 15, 16).

Some subjects have a binocular vision that is strongly dominated by the image perceived by one of their eyes, called the dominant eye. In other words, such subjects have one visual pathway that dominates strongly over the other visual pathway, in the neural process of binocular image fusion. For such subjects, during the binocular refraction protocol described above, a “suppression” phenomenon may occur if a completely blank image is provided to the dominant eye of the subject, due to ocular rivalry between the left and right visual pathways of the subject. In this case, the image perceived by the subject is completely blank.

Such suppression of the optotypes, in the perceived image, makes of course the determination of the refraction error of the non-dominant eye difficult, or even impossible while maintaining a binocular vision. Even if the ocular dominance of the subject is not strong enough to cause such suppression, it often causes blinking or flickering of the image perceived by the subject. Besides, during such a binocular refraction protocol, a flickering of the image perceived by the subject may also be caused by vision problems of the subject related to ocular vergence. These adverse effects make the binocular refraction protocol less accurate, or less comfortable for the subject, and longer to be carried on.

The peripheral part 52 of the test images having peripheral image components stabilizes the fusion of the two test images by the brain of the subject and reduces the “suppression” phenomenon. This way, the visual test is more comfortable for the subject and provides more accurate results.

Advantageously, the test images provided to the eyes of the subject with said optometry device 1 have different contrast. The tested-eye test image provided to the tested eye (with optotypes/target in the central part) has a contrast of more than 80%, preferably more than 90%, preferably equal to 100%. The non-tested-eye test image provided to the other eye, with blank center or with the non-tested-eye target, identical to the tested-eye target but with lower contrast, has a contrast of less than 15%; preferably less than 10%, preferably equal to 5%.

The contrast of any central part with optotype may be defined as:
(Loptotype−Lbackground)/(Loptotype+Lbackground), Loptotype being the luminance of the optotype and Lbackground being the luminance of the background.

Alternatively, in the case where the central part of the non-tested-eye test image comprises the same target than the tested-eye test image, with lower contrast, the peripheral part of the tested-eye test image and the peripheral part of the non-tested-eye test image may have the same contrast. Only the central part of the tested-eye test image and the central part of the non-tested-eye test image have different contrasts. For example, the central part of the tested-eye test image has a contrast of more than 80%, preferably more than 90%, preferably equal to 100%. The central part of the non-tested-eye test image has a contrast of less than 15%; preferably less than 10%, preferably equal to 5%. In a general manner, the central part of the non-tested-eye test image has a contrast of less than 50% of the contrast of the central part of the tested-eye test image.

In a general manner, said non-tested-eye test image is displayed with a contrast inferior to half of the contrast of the tested-eye test image.

Moreover, in order to improve the comfort of the subject and further reduce the suppression effect, the peripheral part 52 of each test image comprises components having different binocular disparities.

Preferably, each component of said plurality of peripheral image components is displayed with a specific disparity between the test images provided to both eyes of the subject that is different from the disparity associated to other components of said plurality of peripheral image components.

In real life, because of the eyes' horizontal separation, the two eyes of the subject have a different point of view on any scene observed by the subject. The difference in the point of view of the eyes creates a binocular disparity that the brain uses to extract depth information from the combination of two two-dimensional retinal images.

Binocular disparity refers to the difference in position of an object seen by the left and right eyes during binocular observation of this object. In practice, it is representative of the distance between two corresponding points in the left and right image of a stereo pair, reflecting the difference in image location of an object seen by the left and right eyes resulting from the eyes' horizontal separation.

A similar difference in point of view is simulated in a stereoscopic 3D representation by creating two images differing from each other by a difference in point of view corresponding to a predetermined disparity, each of them being displayed to one of the eyes of the subject.

The disparity of the stereoscopic 3D representation of any component of said test images is determined based on the actual distance between this object of the subject in real life and on the interpupillary distance of the subject. The disparity is typically larger for objects that are closer to the subject than for the objects that are further away.

It may be expressed as a visual angle under which the component is seen. It may also be calculated as a number of pixels.

For determining the disparity, the size of the screen, the interpupillary distance, the distance between the eye of the subject and the screen are taken into account.

The test images shown on FIGS. 5 to 7 and 9 to 15 are test images as displayed on the screens of the display system. In the test images of the examples shown on FIGS. 5 to 7 and 9 to 15, the components of the peripheral parts of the test images are fruits, vegetables or cooking tools. Each type of fruit, vegetable or cooking tool is displayed with a different disparity. For example, components of the peripheral part of the test images have disparities of 1600″, 800″, 400″, 340″, 280″, 200″ 160″, 120″, 100″, 80″. These disparities are expressed in minutes of arc angles.

The central parts 51 of the test images of the examples shown on FIGS. 5 to 7 each comprises a visual target T1 for spherical refraction determination of the eyes.

FIG. 5 shows the test images I11, I12 for step d1) or d3) where both eyes are tested simultaneously.

FIGS. 6 and 7 show the test images I21, I22, I31, I32 displayed to test a first eye (FIG. 6) separately from the other and the test images provided to test the second eye (FIG. 7). On FIG. 6, a tested-eye test image I21 with a tested-eye target T1 is provided to the first eye and a non-tested eye test image I22 with a non-tested-eye target T1′ identical to test-target T1 with lower contrast than the tested-eye target T1, is provided to the second eye. On FIG. 7, a tested-eye test image I32 with a tested-eye target T1 is provided to the second eye and a non tested-eye test image I31 with a non-tested-eye target T1′ identical to test-target T1 with lower contrast than the tested-eye target T1 is provided to the first eye. The non-tested-eye test images I22 and I31 may be the same as the tested-eye test images I21, I32 with a lower contrast, for example lower than half of the contrast of the tested-eye test image I21, I32. Alternatively, non-tested-eye test images I22 and I31 may be the same as the tested-eye test images I21, I32 with only the central part having lower contrast, for example lower than half of the contrast of the central part of the tested-eye test image I21, I32, the contrast of the peripheral parts of the two test images being the same.

The final image IF seen by the subject in all cases is shown on FIG. 8.

In the example of the figures, the tested-eye test image I21, I32 seen by the tested eye has a contrast equal to 100% and the non-tested-eye target T1′ seen by the non tested eye has a contrast of 5%. The peripheral components of the test images I21, I22, I31, I32 are in 3D (stereo vision) for more comfort and less suppression. These peripheral components have different levels of 3D representation, in other words, different disparities, depending on the component.

FIGS. 9 and 10 show the test images I41, I42 provided to perform step d2) to test a first eye (FIG. 9) separately from the other and the test images I51, I52 provided to perform step d2) for the second eye (FIG. 10). The targets T2, T2′ is a cloud or a matrix of black points on green background. They are here shown at the center of the image of a kiwi to attract the attention of the subject. The peripheral part 52 of each test image comprises here several fruits displayed with different disparities. The non-tested-eye targets T2′ are identical to the tested-eye targets T2 with lower contrast, as described in the case of FIGS. 6 and 7.

FIG. 11 shows test images I61, I62 used in a binocular balance step. The lines of points of the specific targets T3A, T3B are displayed at the center of a cake.

The peripheral part 52 of each test image comprises several ingredients for the cake displayed with different disparities.

FIG. 12 shows test images I71, I72 used in step e). The optotypes of the target T4 are displayed at the center of a book. The peripheral part 52 of each test image comprises several fruits displayed with different disparities.

Similarly, the test images I81, I82, I91, I92, I101, I102 shown on FIGS. 13 to 15 are used for a visual acuity test. The optotypes of the tested-eye target T5 are displayed in the central part 51 of each tested-eye test image I81, I92, I101, I102. The peripheral part 52 of each test image comprises several fruits displayed with different disparities.

The central part 51 of each of the non-tested-eye I82, I91 comprises here no target.

In the method according to the invention, as mentioned before, said test images may be displayed by one or two screens of the display system. Each screen may belong to a smartphone.

Finally, the method according to the invention is implemented by a control unit comprising for example a computer with at least a processor and a memory. The computer may be programmed to display the test images with an appropriate timing.

The sets of test images for first and second eyes may be put in the memory of the computer. The computer may comprise the smartphones when the screens used belong to smartphones. The change from a test image to the next may be manually triggered by the operator of the optometry device, for example by clicking on the screens when he wants to change the image.

Alternatively, said control unit may comprise a smartphone different from the one or two smartphones used in the display system to provide screens.

All smartphones are connected to the same Wi-Fi network, and communicate by TCP-IP protocol. They could also communicate using Nearby API protocol.

A message is sent to each smartphone of the display system 20 indicating the step of the method to be performed.

Each smartphone of the display system receives the message and opens a file with a list of all the test images corresponding to this step. Once this file opens, the first image of the list is displayed on the screen of the corresponding smartphone.

The operator can remain away from the wearer and from the test.

According to the invention, the method for determining a complete set of values of refractive features of an eye of a subject in far and near and/or intermediary vision conditions also comprises a determination 101 of accurate values of refractive features of an eye of a subject in far vision conditions performed before the determination of accurate values of refractive features of an eye of a subject in near and/or intermediary vision conditions as described above.

Advantageously, said determination 101 of accurate values of refractive features of an eye of a subject in far vision conditions comprises steps that are similar to the ones of the method for determining accurate values of refractive features of an eye of a subject in near and/or intermediary vision conditions. They are carried out with the optometry device in its horizontal configuration.

The following steps are performed:
A) adjusting the relative position of the subject and said refraction test unit so that the pupils of his eye are aligned with said first and second optical axes in far vision conditions,
B) determining a vertex distance between said first or second optical refraction element and said first or second eye of the subject in far vision conditions,
C) determining a value of a parameter representative of an accommodation feature of the eyes of the subject in far vision conditions or altering said accommodation of the eyes of the subject,
D) determining preliminary values of said refractive features of the eyes of the subject in far vision conditions, comprising at least a preliminary value of the spherical refraction of each eye and a preliminary value of the cylindrical refraction of each eye,
E) checking binocular visual perception of the subject by adding to the vision correction powers provided to the eyes an added predetermined spherical power,
F) determining a final visual acuity of the subject in near and/or intermediary vision conditions, and
G) determining said accurate values of refractive features of an eye of a subject in far vision based on the results of the previous steps,
wherein in said step d), the following steps are performed in this order:
   D1) measuring a first preliminary value of spherical refraction of each of the eyes of the subject,
   D2) measuring said preliminary value of cylindrical refraction of each of the eyes,
   D3) measuring a second preliminary value of spherical refraction of each of the eyes of the subject, said preliminary value of spherical refraction of each of the eyes of the subject being determined based on said second preliminary values.

The steps are performed as described before, except for the fact that they are adapted to far vision tests. As mentioned before in the description relative the near or intermediate vision test protocol, the preliminary values of the spherical and cylindrical refraction values of the eye in far vision conditions may comprise a spherical power Se and a cylindrical power Ce having an orientation represented by an angle Ae. The preliminary values of the spherical and cylindrical refraction values of the eye may also comprise the values of any other set of parameters representing the above mentioned refractive power features of the eyes, such as a triplet {Me, J0e, J45e} as defined before. During step D2), the equivalent sphere remains constant.

The refractive features of said eye in far and near vision conditions are determined within 24 hours. They are preferably determined on the same day. Preferably, the refractive features of said eye in far vision conditions is determined before determining the refractive features of said eye in near vision conditions.

It is particularly useful to have similar protocols for determining refraction features of the eye of the subject both in far and near/intermediary visions. Since protocols are similar, the risk of having error/deviation between far vision and near/intermediary vision measurements is reduced.

Performing both far vision and near/intermediary tests within a short period also avoids errors/deviations between the two measurements.

Steps A) and B) are performed as steps a) and b) of the method described for near and/or intermediary vision.

Steps B) to F) are performed while the relative position of the subject and said refraction test unit is adjusted according to this step A).

In the method according to the present disclosure, the vertex distance determined in step B) for each eye is kept constant for all the subsequent steps carried out using the refraction test unit 10.

Step C) may be carried out in a way that is similar to the step c) in near/intermediary vision, or it can correspond to a step of fogging the vision of the subject in order to prevent his accommodation. In addition, this step C) is performed while maintaining the vertex distance determined at step B) and at the relative position of the subject and refraction test unit determined at step A).

This step of fogging is performed as known from the state of the art.

For example, the step of fogging uses a test image with a letter or line of letters as an optotype is displayed. If it is read by the subject, the optotype is fogged with a positive spherical power addition.

The initial powers of the lenses of the optical refraction elements 11, 12 are determined based on an objective measured value of the refraction of the eyes, performed for example with auto-refractor or based on the powers of the ophthalmic lenses of the current optical equipment of the subject or on the previous prescription of the subject.

The refractive powers of the lenses of the optical refraction elements 11, 12 are then modified. Positive spherical power is added in front of the eye of the subject until the letter is no longer readable to get the fogging spherical power value for a first target visual acuity, for example 0.3 LogMAR.

Step C) then also comprise a defogging step. Negative or less positive spherical power values are provided to the eye of the subject until the most convex sphere giving the best sharpness is obtained. The positive spherical value added is for example removed in order to obtain a second target visual acuity, for example of about −0.1 LogMAR for a spherical eye without amblyopia. The subject is then asked:

“Can you read the lines of letters in the center of the screen?”

Step C) allows providing a value of the spherical refraction of the eye for which the eye of the subject has a relaxed accommodative vision.

The value of the spherical refraction provided at the end of the step C) is then used as a starting point for step D).

Steps D) is preferably performed as mentioned in near/intermediary vision. For example, in step D1), the initial value of the spherical power of the set of optical refraction elements 11, 12 corresponds to the value of the spherical power allowing to obtain a relaxed accommodative vision as determined in step C). Step D1) is preferably performed as mentioned in the near/intermediary vision.

In another embodiment of step D1), a search for the maximum convex sphere (i.e. the spherical refraction of the eye) is for example carried out with a red/green duo-chrome test. Its goal is to find the sphere which gives the best sharpness, with a final visual acuity target for example equal to −0.1 LogMAR. The target is for example a line of letters on a red/green background. The subject must choose whether he sees the letters blacker on one of the two backgrounds, or if the contrasts of the letters are the same. If possible, this test is performed with a 0 LogMAR acuity line, if the subject can read the entire line of letters, otherwise on a lower acuity line. The question asked to the subject may be:

“Tell me if you find the letters more contrasting on red, green, or if you find them identical”. This embodiment of step D1) provides the first value of the spherical refraction of each of the eyes.

Steps D2) and D3) and the other steps such as steps, E) and F) are performed as mentioned in near/intermediary vision.

As mentioned in the description of the method used in near/intermediary vision, optional steps of preliminary visual acuity test and/or binocular balance may be performed.

Optionally, before step D) or, preferably, between steps B) and C), a preliminary visual acuity test of the eyes of the subject is performed.

This step is performed with far vision conditions corresponding to the vision conditions of the test currently performed.

Each of steps D1), D2) and D3) are preferably performed with binocular vision of the subject with only one eye provided with a test image or both eyes provided with a test image.

Between steps D) and E), a step of adjusting the binocular balance of the eyes of the subject to obtain adjusted values of said refractive features of the eyes in near or intermediary vision conditions is performed.

The binocular balance test may be performed by any known protocol.

In an embodiment, before performing steps a) to g) of the method in near/intermediary vision, the refractive features of the eye in far vision conditions are determined as mentioned before, and a test 102 for determining the need for correcting astigmatism of the eye is performed, comprising measuring the visual acuity of the eye with test images having different contrasts.

Alternatively, the refraction features of the eyes in far vision may be determined based on the current optical equipment or the previous prescription of the subject.

The test for screening and/or determining the need for correcting astigmatism of the eye is performed by measuring the binocular and/or monocular visual acuity in near vision with a high and low level contrast visual acuity target.

For example, a visual acuity target comprising at least a line of optotypes is first provided to the eyes of the subject with a 100% contrast. Then, in a second step, it is provided to the eyes with a contrast comprised between 5 and 10%.

If the visual acuity of the subject decreases with the decrease in contrast, a different target comprising black lines extending radially from a point is provided to both eyes. The subject is asked whether all lines are seen identical. If the subject perceived some lines more black than others, it is determined that the determination of accurate refraction features of his eyes in near and/or intermediary vision would be particularly beneficial.

The comfort of the subject may also be tested with natural images. The reading speed of the subject may also be tested.

In another example, a specific questionnaire can be used to determine the needs of the subject and/or the difficulties of the subject in near and/or intermediary vision conditions.

1. A method for determining accurate values of refractive features of an eye of a subject in near and/or intermediary vision conditions, using an optometry device having a refraction test unit with a first optical refraction element adapted to provide different vision correction powers to a first eye of the subject along a first optical axis and a second optical refraction element adapted to provide different vision correction powers to the second eye of the subject along a second optical axis,
comprising the following steps:
a) adjusting the relative position of the subject and said refraction test unit so that the pupils of his eye are aligned with said first and second optical axes in near and/or intermediary vision conditions;
b) determining a vertex distance between said first or second optical refraction element and said first or second eye of the subject in near and/or intermediary vision conditions;
c) determining a value of a parameter representative of an accommodation feature of the eyes of the subject in near and/or intermediary vision conditions;
d) determining preliminary values of said refractive features of the eyes of the subject in near and/or intermediary vision conditions, comprising at least a preliminary value of the spherical refraction of each eye in near and/or intermediary vision conditions and a preliminary value of the cylindrical refraction of each eye in near and/or intermediary vision conditions,
e) checking binocular visual perception of the subject by providing to the eyes vision correction powers equal to said preliminary values of the corresponding spherical and cylindrical refractions with an added predetermined spherical power;
f) determining a final visual acuity of the subject in near and/or intermediary vision conditions; and
g) determining said accurate values of refractive features of the eye of a subject in near and/or intermediary vision based on the results of the previous steps,
wherein, in said step d), the following steps are performed in this order:
measuring a first value of spherical refraction of each of the eyes of the subject,
measuring said preliminary value of cylindrical refraction of each of the eyes,
measuring a second value of spherical refraction of each of the eyes of the subject, said preliminary value of spherical refraction of each of the eyes of the subject being determined based on said second value.

2. The method according to claim 1, wherein, in step a), a camera oriented towards the eyes of the subject, an eye tracking device or a support for the head of the subject is used.

3. The method according to claim 1, wherein, in step b), at least a profile image of the subject placed in front of the refraction test unit is acquired, and the distance between each eye and the corresponding optical refraction test element is deduced from this profile image.

4. The method according to claim 1, wherein, before step d), a preliminary visual acuity test of the eyes of the subject is performed with initial spherical power and/or cylindrical power and axis of each optical refraction element determined based on refraction features of a current optical equipment of the subject or on the refraction features of the eyes of the subject in far vision conditions.

5. The method according to claim 1, wherein, in step c), a binocular accommodation test is performed by presenting each of the eyes of the subject with an identical target placed at near or intermediary vision optical distances from the eyes of the subject and modifying the spherical powers of each of the first and second optical refraction elements to determine the minimum spherical powers of the first and second optical refraction element for which the subject accommodates on the target.

6. The method according to claim 1, wherein each step of measuring said first value of spherical refraction of each of the eyes of the subject, measuring said preliminary value of cylindrical refraction of each of the eyes, and measuring said second value of spherical refraction is performed by testing both eyes of the subject simultaneously in binocular vision, or by testing each eye of the subject separately from the other while maintaining a binocular vision.

7. The method according to claim 1, wherein said first and second values of spherical refraction and said preliminary value of cylindrical refraction of each of the eyes of the subject are determined by testing each eye separately from the other while maintaining a binocular vision and said first values of spherical refraction, said preliminary value of cylindrical power and said second value of spherical refraction are obtained by alternating measures on the first and second eyes of the subject.

8. The method according to claim 1, wherein said preliminary values of the spherical and cylindrical refraction of the eye comprise a spherical power Se and a cylindrical power Ce having an orientation represented by an angle Ae or an equivalent sphere Me equal to the spherical power Se plus half of the cylindrical power Ce and refractive powers J0e, J45e of two Jackson crossed cylinders lenses representative of the cylindrical refractive power features of the eye.

9. The method according to claim 1, wherein, between steps d) and e), a step of adjusting the binocular balance of the eyes of the subject to obtain adjusted values of said spherical and cylindrical refraction of the eyes in near or intermediary vision conditions is performed, and in step e) said binocular visual perception of the subject is checked by providing the eyes with vision correction powers equal to said adjusted values of spherical and cylindrical refraction with an added predetermined spherical power.

10. The method according to claim 1, wherein, in each step where each of the eyes is tested separately from the other while maintaining a binocular vision:
a tested-eye test image with target is provided to the tested eye, comprising images of realistic activities carried out in near vision having a plurality of peripheral image components and a test target displayed in the center of the test image, and
a non-tested-eye test image is provided to the other eye comprising similar images of realistic activities carried out in near vision having a plurality of peripheral image components and the same test target than displayed on the tested-eye test image with a contrast inferior to the contrast of the test target of the tested-eye test image, the peripheral image component of the two test images being stereoscopically displayed.

11. The method according to claim 10, wherein in each step where both eyes are tested simultaneously, a test image with target is provided to each eye, comprising images of realistic activities carried out in near or intermediary vision having a plurality of peripheral image components displayed stereoscopically,
each component of said plurality of peripheral image components being displayed with a specific disparity between the images provided to both eyes of the subject that is different from the disparity associated to other components of said plurality of peripheral image components.

12. The method according to claim 1, wherein before performing steps a) to g), the refractive features of the eye in far vision conditions are determined, and a test for determining the need for correcting astigmatism of the eye is performed, comprising measuring the visual acuity of the eye with test images having different contrasts.

13. The method according to claim 1, wherein during said steps a) to f), said first and second optical axes of said optical refraction elements are inclined downwards.

14. A method for determining a complete set of values of refractive features of an eye of a subject in far and near and/or intermediary vision conditions, comprising a determination of accurate values of refractive features of an eye of a subject in far vision conditions and a determination of accurate values of refractive features of an eye of a subject in near and/or intermediary vision conditions according to claim 1.

15. The method according to claim 14, wherein said determination of accurate values of refractive features of an eye of a subject in far vision conditions comprises the following steps:
A) adjusting the relative position of the subject and said refraction test unit so that the pupils of his eye are aligned with said first and second optical axes in far vision conditions;
B) determining a vertex distance between said first or second optical refraction element and said first or second eye of the subject in far vision conditions;
C) determining a value of a parameter representative of an accommodation feature of the eyes of the subject in far vision conditions or altering said accommodation of the eyes of the subject;
D) determining preliminary values of said refractive features of the eyes of the subject in far vision conditions, comprising at least a preliminary value of the spherical refraction of each eye and a preliminary value of the cylindrical refraction of each eye;
E) checking binocular visual perception of the subject by adding to the preliminary value of the spherical refraction an added predetermined spherical power;
F) determining a final visual acuity of the subject in near and/or intermediary vision conditions; and
G) determining said accurate values of refractive features of an eye of a subject in far vision based on the results of the previous steps, wherein in said step d), the following steps are performed in this order:
measuring a first preliminary value of spherical refraction of each of the eyes of the subject,
measuring said preliminary value of cylindrical refraction of each of the eyes,
measuring a second preliminary value of spherical refraction of each of the eyes of the subject, said preliminary value of spherical refraction of each of the eyes of the subject being determined based on said second preliminary values.
